(19) United States
(12) Patent Application Publication
(10) Pub. No.: US 2024/0252364 A1
(43) Pub. Date: Aug. 1, 2024

(54) Reusable Swim Diaper

(71) Applicant: O2COOL, LLC, Chicago, IL (US)

(72) Inventor: Becky Cannon, Asheville, NC (US)

(73) Assignee: O2COOL, LLC, Chicago, IL (US)

(21) Appl. No.: 18/189,313

(22) Filed: Mar. 24, 2023

Related U.S. Application Data
(60) Provisional application No. 63/482,387, filed on Jan. 31, 2023.

(51) Int. Cl. A61F 13/49

(52) U.S. Cl. CPC A61F 13/49006; A61F 2013/15195

(57) ABSTRACT

A reusable swim diaper having a main body made of waterproof coated (bonded polyurethane) polyester configured with an elasticized waist opening and elasticized leg openings, the main body having improved degradation from laundering and is suitable for multiple launderings and reuse without degrade and having gussets at the leg openings to minimize the flow of particulate matter, such as fecal matter, between the inside and the outside of the swim diaper.

### FIELD

The present disclosure relates to reusable swim diapers for infants and young children. More particularly, the disclosure relates to reusable swim diapers of improved materials and with gussets of improved material to provide a snug fit and add protection against pool accidents.

### BACKGROUND

Improvement is desired in the provision of reusable swim diapers. What is desired is a diaper having improved construction that minimize the flow of particulate matter, such as fecal matter, between the inside and the outside of the swim diaper.

### SUMMARY

The disclosure advantageously provides a reusable swim diaper having improved construction and materials to minimize the flow of particulate matter, such as fecal matter, between the inside and the outside of the swim diaper.

In one aspect, a swim diaper according to the disclosure includes a main body made of a waterproof coated bonded polyurethane polyester material including a composite material made of one or more layers of polymer resins joined by urethane links and a polyester backing, the main body configured with an elasticized waist opening and elasticized leg openings including gussets interior of the leg openings also made of the waterproof coated bonded polyurethane polyester material of the main body.

The main body has improved degradation from laundering and is suitable for multiple launderings and reuse without degrade, and together the material of the main body and the gussets serve to minimize the flow of fecal matter between the inside and the outside of the swim diaper.

### BRIEF DESCRIPTION OF THE DRAWINGS

Further advantages of the disclosure are apparent by reference to the detailed description when considered in conjunction with the figures, which are not to scale so as to more clearly show the details, wherein like reference numbers indicate like elements throughout the several views, and wherein:

FIG. 1 shows a reusable swim diaper according to the disclosure.

FIG. 2 shows an exterior of the swim diaper.

FIG. 3 shows an interior of the swim diaper.

FIG. 4 is a close-up of a gusset feature of the diaper.

### DETAILED DESCRIPTION

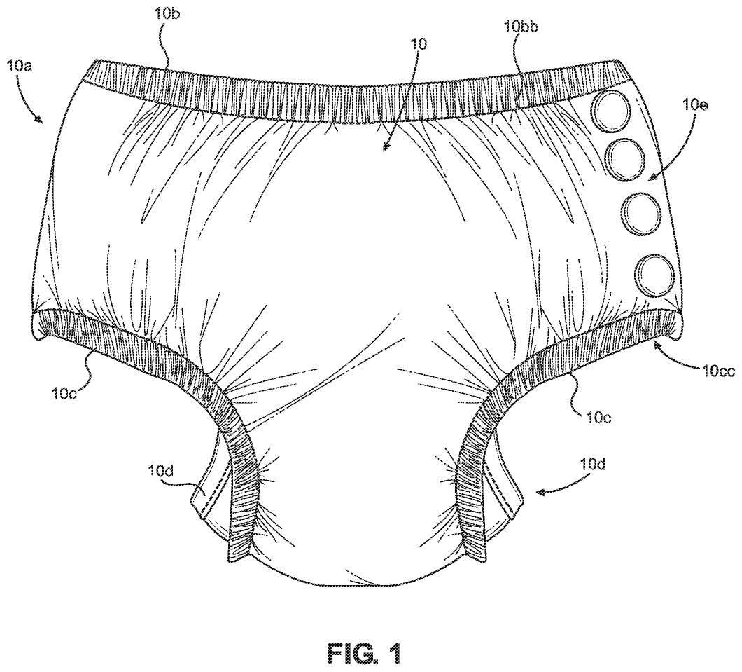

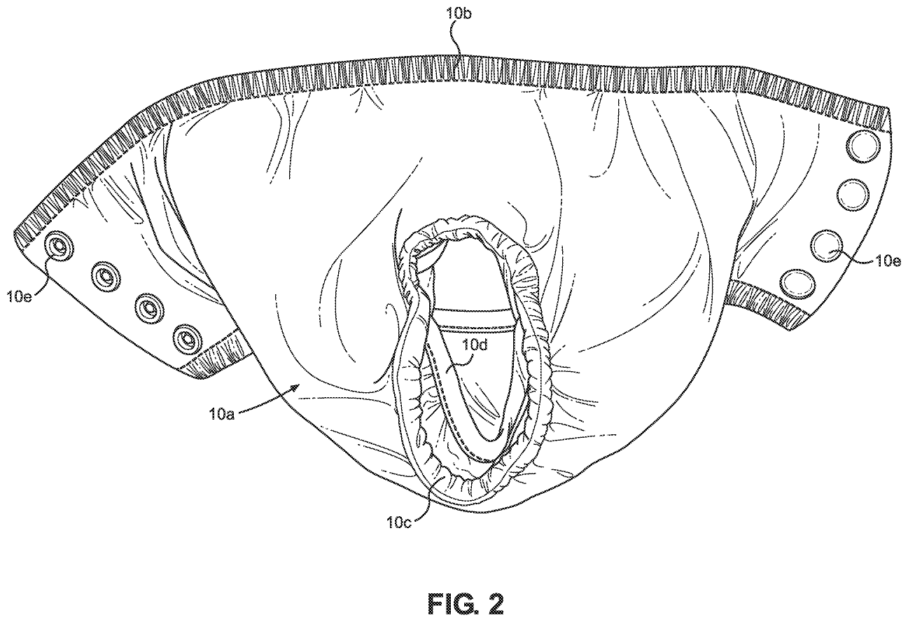

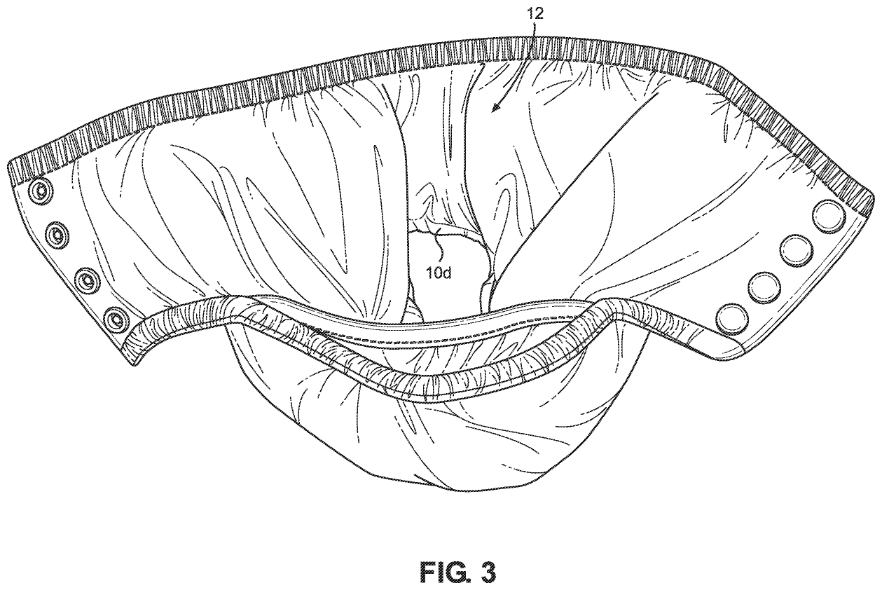

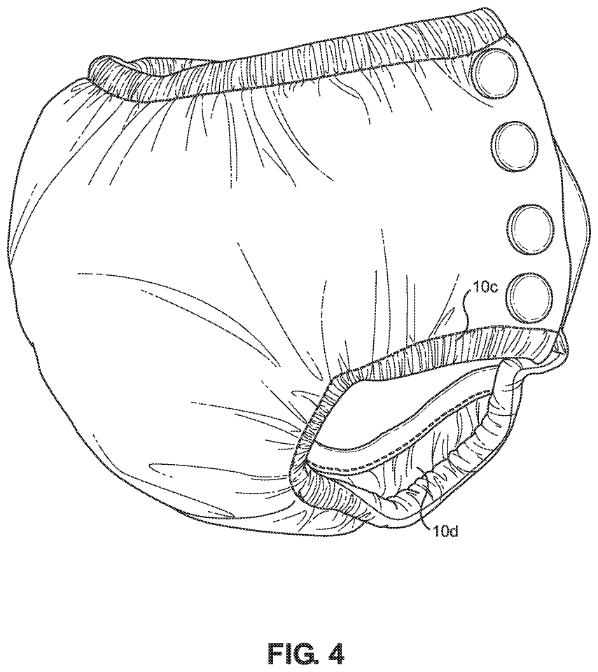

With reference to FIGS. 1-4 the disclosure provides a reusable swim diaper 10 having improved construction by virtue of improved materials and improved features.

The diaper 10 is made of waterproof coated bonded polyurethane knitted polyester material. More specifically, the waterproof coated bonded polyurethane knitted polyester material is a composite material made of one or more layers of polymer resins joined by urethane links and a knitted polyester backing. This material has been observed to provide improved durability and performance for use as a reusable swim diaper. This material has been observed to have improved degradation from laundering and is suitable for multiple launderings and reuse without degrade.

The diaper 10 has a one-piece main body 10a configured to include an elasticized waist 10b and elasticized leg openings 10c. The waist 10b is formed with a waist seam 10bb and the leg openings 10c have leg opening seams 10cc. Gussets 10d are located at the leg openings 10c. Snaps 10e are provided for snugly securing the waist 10b around the waist of the baby.

The waist seam 10bb is made by sewing elastic on the inside of the waist 10b in a manner to provide comfort seams. The comfort seams are formed by turning both edges of the waist material inside and sewing them together so the rough end edges of the material are concealed within the seam and not exposed to rub the waist of the baby.

The leg opening seams 10cc are made in the same manner with comfort seams formed by turning both edges of the leg opening material inside and sewing them together so the rough end edges of the material are concealed within the seam and not exposed to rub the leg of the baby.

The gussets 10d are made of the same waterproof coated (bonded polyurethane) knitted polyester material as the main body 10. The gussets 10d are made in the same manner as the comfort seams 12 and 14, with rough edges turned inside and sewn together so that no rough edges are exposed. The gussets 10d have been observed to enable a snug fit and add protection against pool accidents.

The exterior surface of the polyester material of the diaper 10 may be printed with ornamental and decorative features. The interior surface of the polyester material of the diaper 10 desirably has an absorbent material 12, such as terrycloth, bonded thereto. The absorbent material 16 is desirably two layers of terrycloth provided as by a polyester/polyamide fiber blend.

The construction of the diaper 10 including the materials utilized, the elastic apertures at the waist and leg openings, and the additional gussets 10d, serve to minimize the flow of particulate matter, such as fecal matter, between the inside and the outside of the swim diaper between the inside and the outside of the swim diaper 10

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated.

1. A reusable swim diaper configured for retaining fecal matter, comprising: a main body made of a waterproof coated bonded polyurethane polyester material comprising a composite material made of one or more layers of polymer resins joined by urethane links and a polyester backing, the main body configured with an elasticized waist opening and elasticized leg openings including gussets interior of the leg openings also made of the waterproof coated bonded polyurethane polyester material of the main body, wherein the main body has improved degradation from laundering and is suitable for multiple launderings and reuse without degrade, and together the material of the main body and the gussets serve to minimize the flow of fecal matter between the inside and the outside of the swim diaper.

2. The reusable swim diaper of claim 1, wherein the polyurethane polyester material is a polyurethane knitted polyester material and the polyester backing is a knitted polyester backing.
